(19) United States
(12) Patent Application Publication
(10) Pub. No.: US 2013/0296814 A1
(43) Pub. Date: Nov. 7, 2013

(54) Carry Belt for Vacuum Surgical Drains

(71) Applicant: Joanie Antholz, Duncan, OK (US)

(72) Inventor: Joanie Antholz, Duncan, OK (US)

(21) Appl. No.: 13/462,806

(22) Filed: May 2, 2012

(51) Int. Cl. A61M 1/00

(52) U.S. Cl. 604/319

(57) ABSTRACT

The present invention relates to a post-operative device to support and enclose vacuum surgical drains such as Jackson-Pratt® drains while facilitating maintenance of the drains and accurate record keeping by medical personnel. A containing element consisting of a plurality of sub-pouches each for holding a single drain is provided. In one embodiment the containing element is affixed to the body by a strap. In a second embodiment, the containing element is affixed to the body by means of an adhesive pad. In both embodiments, unique identifying marks are provided on the exterior aspect of each sub-pouch to facilitate accurate record keeping by medical personnel.

### CROSS-REFERENCE TO RELATED APPLICATIONS

None.

### FIELD OF THE INVENTION

The present invention relates to a belt that facilitates the use of vacuum surgical drains generally and to a belt that facilitates carrying, storing, emptying, and assessing Jackson-Pratt® and other related vacuum surgical drains specifically.

### BACKGROUND OF THE INVENTION

Vacuum surgical drains, particularly Jackson-Pratt® (J-P) vacuum surgical drains, are widely used in the post-operative care of surgical patients around the world. A J-P drain is comprised of a small, translucent plastic or rubber bulb attached to a length of drain tube. The open, free end of the drain tube is insinuated in the surgical wound before the surgical field is closed while the drain bulb remains outside the patient's body. Air is evacuated from the drain bulb by first opening a discharge port, gently squeezing the bulb to expel the air inside, and closing the discharge port. Over time, the bulb expands slowly and as it does it creates a slight vacuum in the drain tube. By means of this vacuum, excess fluid present in the surgical wound is evacuated into the bulb. When the bulb is full, or on a regular schedule, a nurse or technician opens the discharge port, empties the bulb through the discharge port, recompresses the bulb, closes the discharge port, and the cycle repeats. When emptying the bulb, the nurse or technician ordinarily observes the type and character of the fluid collected. For example, the nurse or technician usually takes note of the color, odor, consistency (serous, serosanguineous, and sanguineous), and amount of fluid and notes this data in the patient's chart. Depending on the type of surgical procedure, a multiplicity of drains may be installed in one surgical wound and the drains may be installed for periods of time ranging from a few days or even few weeks.

Two problems commonly arise when dealing with such drains in actual use: First, for at least some period during the few days or weeks the drains are installed, the patient is ambulatory. Thus, the matter of storing the drains when the patient moves about is thus a common problem. Second, some surgical wounds require numerous drains and unsurprisingly all the drains look the same because they are usually the same model. Thus, when recording the type and character of the fluid collected from each drain, the nurse or technician may become confused and not consistently record the data for the same drain in the same sequence on the chart. Thus during a first emptying, one nurse might record the data for the leftmost drain first and the rightmost drain last while on a subsequent shift a second nurse might record the data for the rightmost drain first and the leftmost drain last. Maintaining a precise and consistent order for recording the type and character of fluid collected is important because as the surgical wound heals, infection may take hold in only one area of the wound with one of the best indicators of the underlying infection being a change in the amount or quality of drainage collected by one particular drain. Thus, if the characteristics of the fluid collected from each drain are not consistently recorded in the same order, medical personnel will be unable to determine the actual site of the infection. Medical personnel and patients have devised a number of solutions to both of these problems.

As to the first problem, medical personnel often pin the drains to the inside of the patient's hospital gown. This works poorly at best because the gown is not attached to the patient and thus the drains may become displaced from the area of the surgical wound potentially unseating the drains as the patient moves about. This is particularly problematic when the patient is bedridden and turns in bed. When the patient is ambulatory, the simple act of bathing or using the toilet becomes a difficult chore because care must be used to ensure that the drains are not moved when lifting the patient's gown. Because of these shortcomings, alternate means of securing the drains have been used. For example, surgical tape may be used to physically tape the drain to the patient, but this is uncomfortable and renders it difficult to empty the drain without first untaping it. Various devices have been disclosed to deal with this problem. For example, U.S. Pat. No. 6,273,872 discloses a belt used to support, among other things, vacuum surgical drains. The principle difficulty with this device is that the drains are exposed and thus prone to becoming entangled in clothing. Also, this device does nothing to facilitate the record keeping necessary when medical personnel manage a patient with multiple drains. U.S. Design Pats. D437,410 and D488,227 both disclose a single pouch device that are used to contain “drain tubes” but only the former appears to be worn about the body. D311,453 discloses a generic bag to be worn about the body which obviously may be employed to hold surgical drains. However, none of these devices provide means of organizing multiple surgical drains nor do they do anything to facilitate record keeping by medical personnel when dealing with a patient with multiple drains. After discharge from the hospital, patients themselves suffer from the same problems and have devised a number of impromptu methods of dealing with J-P drains. For example, kitchen aprons with multiple pouches, loose blouses or shirts with pockets worn inside-out, and elastic bands to which the drains are pinned or taped have all been used to organize and carry J-P drains.

As to the second problem, medical personnel use various ad-hoc means of identifying a particular drain from a multiplicity of drains installed in a surgical wound or wounds. For example, a nurse or technician might write a unique identifier in indelible marker directly on drain bulb, on the tape strip attaching the drain bulb to the patient's gown, on the gown itself, or even on the patient's skin. When assessing the condition of each drain, the nurse or technician uses the unique identifier so assigned to record the information regarding particular drains appropriately in the patient's chart.

But, nothing in the prior art solves all these problems. Because of this, realizing a device that allows for the convenient storage and maintenance of the drains as the patient recovers and becomes ambulatory while aiding medical personnel in their routine data collection and recording tasks would be of great utility.

### BRIEF DESCRIPTION OF THE DRAWINGS

FIG. 1 is a front view of an improved carry belt for vacuum surgical drains capable of carrying two Jackson-Pratt® drains.

FIG. 2 is a cutaway front view of the left sub-pouch (when viewed from the front) of an improved carry belt for vacuum surgical drains showing the inside of the sub-pouch.

FIG. 3 is a top view of the left sub-pouch (when viewed from the front) of an improved carry belt for vacuum surgical drains showing the inside of the sub-pouch.

### SUMMARY OF THE INVENTION

An improved carry belt for vacuum surgical drains in general and Jackson-Pratt® (J-P) drains in particular is disclosed wherein the belt is comprised of a carrying part and an attaching part for attaching the carrying part to some part of the patient's body and holding the carrying part thereto. The attaching part can be: 1) Any kind of strap for circumferentially encircling the patient using any equivalent means of closing and adjusting the length of the strap; or, 2) A paper-backed adhesive pad applied to the back of the carrying part. The carrying part includes a plurality of sub-pouches each equipped with a means for securing a J-P drain bulb inside the sub-pouch and a means for closing the sub-pouch with the drain bulb inside. The attaching part and carrying part are comprised of any mix of flexible fabric, mesh, or netting and are sterilizable. Finally, distinguishing letters or numbers or other symbols are printed on the outside of each sub-pouch to facilitate accurate record keeping by medical personnel when the drain bulbs inside are emptied.

In use, the improved carry belt for J-P drains is affixed to the patient's body below the level of the surgical incision. This can be done using a strap circumferentially encircling the patient's body or by means of a paper-baked adhesive pad affixed to the back surface of the carrying part. The J-P drains installed by the physician during surgery are then each placed in a different sub-pouch and the sub-pouch is closed. Periodically, medical personnel inspect the drains and empty them one at a time, noting the type and character of fluid collected. This information is recorded in the patient's chart using the letter or number or other symbol printed on the outside of the sub-pouch as an identifier for the data. The bulb is returned to the same sub-pouch and the cycle repeats

The present invention represents an improvement over the prior art in several important respects. First, the ability to attach the belt at a particular location below the surgical wound and consistently maintain it there ensures that gravity works to help provide better drainage of the wound. Second, by enclosing the drains in sub-pouches affixed to the patient's body, the drains and the tubes leading to them are physically fixed in the same location with respect to the surgical wound and thus less susceptible to becoming pulled and inadvertently removed from the wound as the patient moves. Finally, by printing a unique identifying letter or number or other symbol on the outside of each sub-pouch, medical personnel now have a convenient means of consistently recording data concerning the type and character of fluid collected.

### DETAILED DESCRIPTION OF THE INVENTION

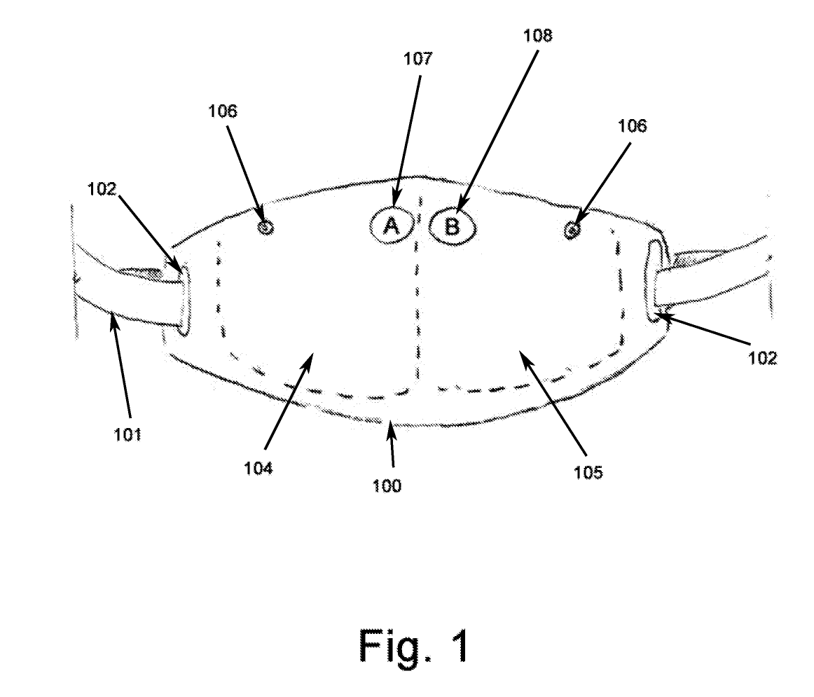

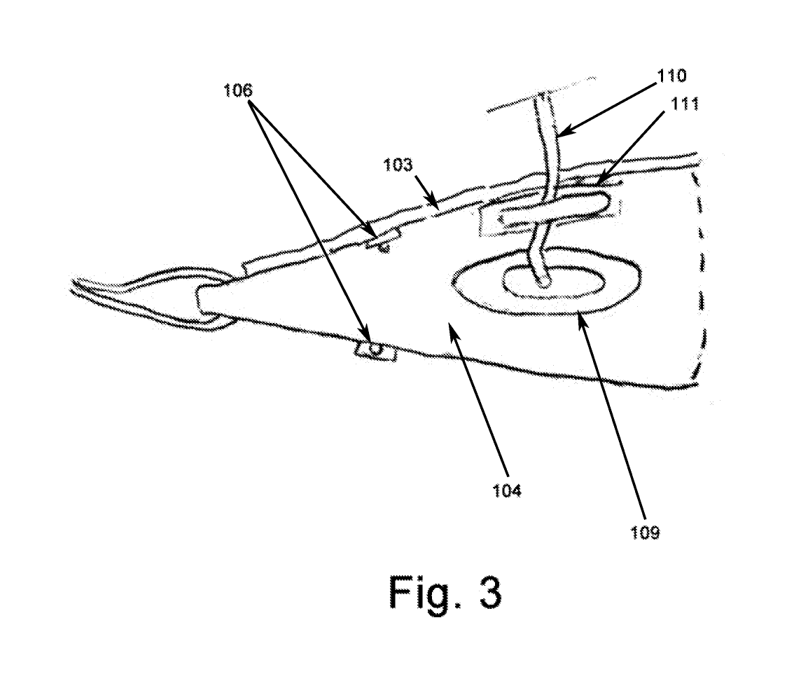

Referring now to FIGS. 1 and 3, an improved carry belt for Jackson-Pratt® (J-P) vacuum surgical drains is comprised of a carrying part 100 and an attaching part. The attaching part may be: 1) A strap 101 for circumferentially encircling the patient at a suitable site below the installed drains; or, 2) A paper-backed adhesive pad 103 affixed to the back panel of carrying part 100. Carrying part 100 is constructed of equal sized rectangular or ovoid front and back panels. To assemble carrying part 100, the front and back panels are placed one atop the other and then joined at their respective peripheral edges by sewing, sonic welding, or equivalent means, leaving the top edge of the each respective front and back panel open thus forming a pouch. This pouch is further sub-divided by sewing, sonic welding, or equivalent means into at least two sub-pouches 104 and 105. Parallel slots 102 formed in the respective ends of carrying part 100 are included for receiving the respective opposite ends of strap 101 if so desired. Strap 101 is constructed with a fastening means affixed at each end, so that if desired, each end of strap 101 may be passed through a different one of two parallel slots 102 and fastened back on itself, thus securing the strap to itself and providing a convenient means of adjusting the length of strap 101 to allow the device to be worn by different sized patients or on different diameter extremities. Ideally, hook and loop fasteners would be used for this task, but those having skill in the art can conceive of numerous equivalent means for accomplishing the same goal. Strap 101 and carrying part 100 may be further comprised of any mix of fabric, mesh, or netting and may be sterilized by any means, including but not limited to: 1) Ethylene oxide gas; 2) Gamma radiation; and, 3) Exposure to electron beams. Mesh or netting used to construct all or part of the exposed front panel of each of sub-pouches 104 and 105 is particularly advantageous in that the drains contained within may be inspected without first removing them. Sub-pouches 104 and 105 are each closeable at the top by means of snap 106 or an equivalent closing means. Affixed to the exposed front panel of each of sub-pouches 104 and 105 are distinguishing visual marks, in this example sequential letters 107 and 108. These distinguishing visual marks are, of course, not limited to letters, but may in fact be any unique combination of numbers, letters, and symbols.

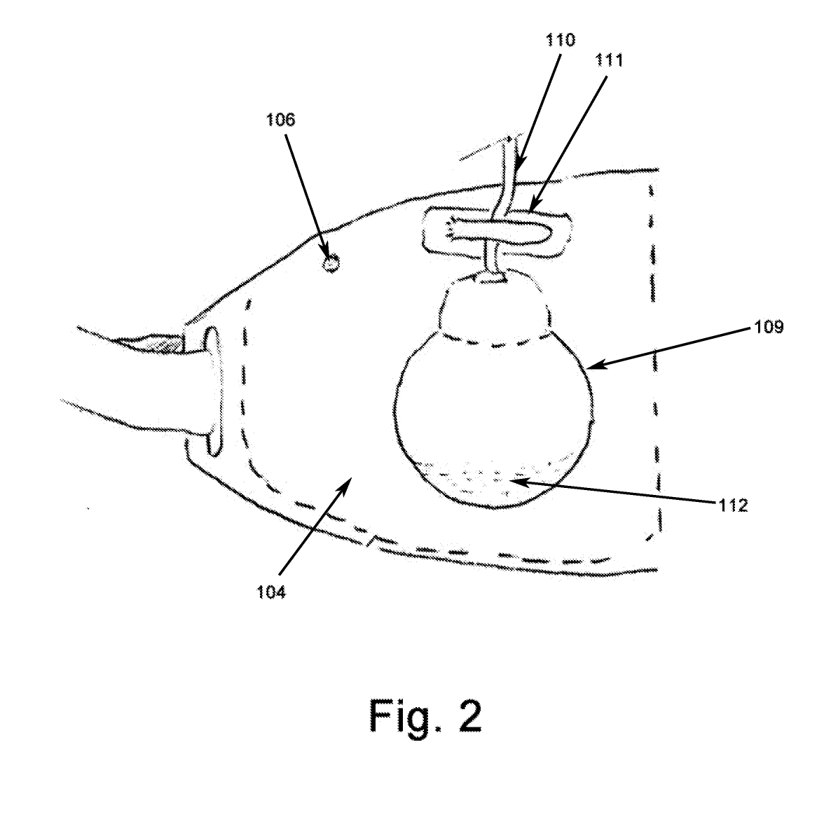

Turning now to FIG. 2, a cutaway view of sub-pouch 104 with compressed drain bulb 109 insinuated within is presented. Drain bulb 109 is connected to drain tube 110. The distal end of drain tube 110 (not shown) is inserted in the closed surgical wound. Hook-and-loop catheter tube holder 111 is affixed inside sub-pouch 104 to the inward facing surface of the back panel of sub-pouch 104 and provides a convenient means of securing drain tube 110 and attached drain bulb 109 inside sub-pouch 104. Sub-pouch 104 is closeable at the top by means of snap 106 or an equivalent closing means. The cutaway view of sub-pouch 105 (not shown) is the reverse mirror image of this view.

Turning now to FIG. 3, a top view of sub-pouch 104 in the open condition with compressed drain bulb 109 insinuated within is presented. Drain bulb 109 is connected to drain tube 110. The distal end of drain tube 110 (not shown) is inserted in the closed surgical wound. Hook-and-loop catheter tube holder 111 is affixed inside sub-pouch 104 to the inward facing surface of the back panel of sub-pouch 104 and provides a convenient means of securing drain tube 110 and attached drain bulb 109 inside sub-pouch 104. Sub-pouch 104 is closeable at the top by means of snap 106 or an equivalent closing means. The top view of sub-pouch 105 in the open condition (not shown) is the reverse mirror image of this view.

Referring now to FIGS. 2 and 3, as evacuated drain bulb 109 slowly expands, a gentle suction is applied to drain tube 110 and thence to a localized area inside the surgical wound. Coupled with gravity, this gentle suction drains excess fluid from the surgical wound and draws it into drain tube 110 and thence into drain bulb 109. Simultaneously, the same process occurs in sub-pouch 105 (not shown).

Referring now to FIGS. 1, 2, and 3, to use the invention carrying part 100 is affixed to the patient's body at an appropriate point below the level of the surgical incision. Medical personnel may affix carrying part 100 in one of two ways: First, each end of strap 101 may be passed through a different one of two parallel slots 102 formed in the left and right ends of carrying part 100 and folded back and fastened on itself, thus securing strap 101 and providing a convenient means of adjusting the length of strap 101 to allow the device to be worn by different sized patients or on different diameter extremities. Second, the paper backing protecting the adhesive surface of paper-backed adhesive pad 103 affixed to the back surface of carrying part 100 may be removed, thus allowing carrying part 101 to be affixed directly to the patient's body without using strap 101. Next, drain bulb 109 associated with a particular J-P drain installed by the physician during surgery is then placed in sub-pouch 104. Drain line 110 associated with drain bulb 109 is then secured inside sub-pouch 104 using hook-and-loop catheter tube holder 111 mounted inside sub-pouch 104. Sub-pouch 104 is then closed by means of snap 106. This process is repeated for the next J-P drain installed by the physician, this time using sub-pouch 105, and so on. Over time, of course, drain bulb 109 expands and fills with fluid 112. At this time, or at periodic times, a nurse or technician disengages snap 106 and releases hook-and-loop catheter tube holder 111 to remove drain bulb 109 from sub-pouch 104. The nurse or technician then assesses the type and character of the fluid, including, without requirement or limitation, the color, odor, consistency (serous, serosanguineous, and sanguineous), and the amount of fluid. This data is noted and recorded in the patient's chart identified by the distinguishing numbers or letters or symbols affixed to the front of sub-pouch 104 from which drain bulb 109 was removed. The nurse or technician then empties drain bulb 109 by manually compressing it, returns compressed drain bulb 109 to sub-pouch 104, reengages hook-and-loop catheter tube holder 111, and reengages snap 106. This process is repeated for the next J-P drain installed by the physician, this time using sub-pouch 105, and so on.

While the above disclosure relates primarily to a device for carrying and maintaining Jackson-Pratt® brand vacuum surgical drains, other types of vacuum surgical drains are equally well supported. For example, the present invention is equally well suited for use with smaller Hemovac® and Davol® brand drains. Also, while the present invention has been described in connection with what are considered to be the most useful and practical embodiments, those having skill in the art will readily discern alternative equivalent configurations and those are to be liberally included within the scope and spirit of the present disclosure. Variations of the present invention featuring more than two sub-pouches are clearly comprehended by the teachings of the present invention. Moreover, variations in which the outer facing surface of the front panel is constructed from a material which may be written upon to record data are also comprehended by the present teaching. These and other variations and are to be included in the scope of the present invention.

What is claimed is:

1. An improved carry belt for vacuum surgical drains comprising:
a) a carrying part used to enclose and carry vacuum surgical drains;
i. wherein said carrying part further comprises a back panel defining the rear surface of said carrying part and a front panel defining the front surface of said carrying part;
ii. wherein said back panel and said front panel are continuously joined along their respective peripheral edges leaving a segment of said peripheral edges of said back panel and said front panel at their respective tops unjoined thus forming a pouch that is open at the top between said back panel and said front panel;
iii. wherein said carrying part further comprises two parallel slots, one at each end, each slot oriented so that it lies perpendicular to the long axis of said carrying part;
iv. wherein said pouch is subdivided into at least two equal sized sub-pouches each of which is open at the top, one for each vacuum surgical drain to be carried;
v. wherein each of said sub-pouches further comprises a means for partially closing the top of said sub-pouch;
vi. wherein each of said sub-pouches is uniquely identified by a distinguishing visual mark affixed to the outward facing aspect of said front panel associated with each of said sub-pouches; and,
b) an attaching part for attaching said carrying part to the patient's body.

2. An improved carry belt for vacuum surgical drains of claim 1 wherein said attaching part is a strap wherein each end of said strap has affixed to it a means of closing and securing said strap to itself.

3. An improved carry belt for vacuum surgical drains of claim 1 wherein said attaching part is a paper-backed, adhesive pad affixed to the outward facing aspect of said back panel of said carrying part.

4. An improved carry belt for vacuum surgical drains of claim 1 wherein said front panel and said back panel are comprised of a material chosen from a group consisting of:
a) fabric;
b) mesh; and,
c) net.

5. An improved carry belt for vacuum surgical drains of claim 1 wherein said carrying part and said attaching part are sterilizable, said sterilization being accomplished by a means chosen from a group consisting of:
a) ethylene oxide sterilization;
b) gamma ray sterilization; and,
c) electron beam sterilization.

6. An improved carry belt for vacuum surgical drains of claim 1 wherein said distinguishing visual marks are from a group consisting of:
a) sequential letters;
b) sequential numbers; and,
c) unique symbols.

7. An improved carry belt for vacuum surgical drains of claim 1 wherein a hook-and-loop catheter tube holder is affixed inside each of said sub-pouches at the top of the inward facing aspect of said back panel associated with each of said sub-pouches.

8. An improved carry belt for vacuum surgical drains of claim 1 wherein said vacuum surgical drains are Jackson-Pratt® surgical drains.

9. An improved carry belt for vacuum surgical drains of claim 1 wherein said vacuum surgical drains are Davol® surgical drains.

10. An improved carry belt for vacuum surgical drains of claim 1 wherein said vacuum surgical drains are Hemovac® surgical drains.

11. A method of using the improved carry belt for vacuum surgical drains of claim 1 comprising the steps of:
a) attaching said carrying part on the patient's body at a suitable point below the surgical incision;
b) placing each one of a multiplicity of said drain bulbs each one of which is associated with one of the multiplicity of said vacuum surgical drains in a different one of said sub-pouches;
c) periodically removing each of said drain bulbs from said sub-pouch in which it was placed and assessing the type, character, and amount of fluid contained within said drain bulb before draining said drain bulb;
d) recording in the patient's chart various notations describing the fluid contained within said drain bulb, said chart notations identified by said distinguishing visible marks affixed to the outward facing aspect of said front panel associated with said sub-pouch from whence said drain bulb was removed; and,
e) replacing each of said drain bulbs in the same one of said sub-pouches from whence it was removed.
